(19) United States
(12) Patent Application Publication
(10) Pub. No.: US 2022/0361805 A1
(43) Pub. Date: Nov. 17, 2022

(54) COGNITIVE LEVEL EVALUATION SYSTEM, COGNITIVE LEVEL EVALUATION METHOD, AND PROGRAM

(71) Applicant: Panasonic Intellectual Property Management Co., Ltd., Osaka (JP)

(72) Inventors: Kengo ABE, Nara (JP); Yoshihiro MATSUMURA, Osaka (JP); Hirobumi NAKAJIMA, Kyoto (JP)

(21) Appl. No.: 17/764,938

(22) PCT Filed: Jun. 8, 2020
(86) PCT No.: PCT/JP2020/022530

§ 371 Date: Mar. 29, 2022

(30) Foreign Application Priority Data
Sep. 30, 2019 (JP) 2019-180749

(51) Int. Cl. A61B 5/00; A61B 5/11

(52) U.S. Cl. CPC A61B 5/4088; A61B 5/1113

(57) ABSTRACT

A cognitive level evaluation system includes a detector and a cognitive level evaluator. The detector is configured to detect a behavior of a subject in a toilet space. The cognitive level evaluator is configured to evaluate a cognitive level of the subject based on a detection result of the detector.

### CROSS-REFERENCE OF RELATED APPLICATIONS

This application is the U.S. National Phase under 35 U.S.C. § 371 of International Patent Application No. PCT/JP2020/022530, filed on Jun. 8, 2020, which in turn claims the benefit of Japanese Patent Application No. 2019-180749, filed on Sep. 30, 2019, the entire disclosures of which applications are incorporated by reference herein.

### TECHNICAL FIELD

The present disclosure generally relates to cognitive level evaluation systems, cognitive level evaluation methods and programs, and more particularly relates to a cognitive level evaluation system configured to evaluate a cognitive level of a subject based on a behavior of the subject, a cognitive level evaluation method and a program.

### BACKGROUND ART

Conventionally, there is known a diagnostic device to diagnose whether or not a subject has a memory disturbance, etc. (e.g., see Patent Literature 1).

The higher brain dysfunction diagnostic device disclosed in Patent Literature 1 includes two operation input means, an instruction means and a reaction time measuring means. The two operation input means each accept the operation from the subject. The instruction means instructs the subject which of the two operation input means should be operated. The reaction time measuring means measures a reaction time, which is a time lag between a time point at which the instruction means instructs the subject and a time point at which any of the two operation input means accepts the operation from the subject. Also, the reaction time measuring means measures the reaction time under a condition that a first arrangement and a second arrangement are incompatible. The first arrangement corresponds to a relative arrangement of the two operation input means (to be operated by the subject) with respect to the center of the subject's body. The second arrangement corresponds to a relative arrangement of the two operation input means with respect to the center of the subject's body parts used when the subject operates the two operation input means.

According to the above configuration, the higher brain dysfunction diagnostic device in Patent Literature 1 can determine whether or not the subject has the memory disturbance, namely evaluate a cognitive level of the subject.

Regarding a person with the potential for dementia, behavioral changes may occur in one aspect of life. In recent years, it is desired that the cognitive level of the person is evaluated based on the person's behavior in one aspect of life. However, in the case of Patent Literature 1, the subject is required to perform the certain operation for the diagnostic device determining whether or not the subject has the memory disturbance. It is hard to say that such the operation is the behavior in one aspect of life.

### CITATION LIST

### Patent Literature

Patent Literature 1: JP 2004-135824 A

### SUMMARY OF INVENTION

It is therefore an object of the present disclosure to provide a cognitive level evaluation system, a cognitive level evaluation method and a program, which can realize evaluating a cognitive level of a subject based on a behavior of the subject in one aspect of life.

A cognitive level evaluation system according to an aspect of the present disclosure includes a detector and a cognitive level evaluator. The detector is configured to detect a behavior of a subject in a toilet space. The cognitive level evaluator is configured to evaluate a cognitive level of the subject based on a detection result of the detector.

A cognitive level evaluation method according to an aspect of the present disclosure includes a detecting step and a cognitive level evaluating step. The detecting step includes detecting a behavior of a subject in a toilet space. The cognitive level evaluating step includes evaluating a cognitive level of the subject based on a detection result in the detecting step.

A program according to an aspect of the present disclosure is designed to cause a computer to execute the cognitive level evaluation method described above.

### BRIEF DESCRIPTION OF DRAWINGS

FIG. 1 is a block diagram illustrating a configuration of a cognitive level evaluation system according to an exemplary embodiment and a configuration of an evaluating system including the cognitive level evaluation system;

FIG. 2 is an appearance drawing for explaining an appearance of a toilet device using a cognitive level evaluation system according to an exemplary embodiment;

FIG. 3 is an appearance drawing for explaining an appearance of an operation device included in the evaluating system;

FIG. 4 is a flowchart for explaining operation about how the cognitive level evaluation system operates;

FIG. 5 is a flowchart for explaining an evaluation processing to be performed by the cognitive level evaluation system; and

FIG. 6 is a drawing for explaining a specific example about how the cognitive level evaluation system operates.

### DESCRIPTION OF EMBODIMENTS

An exemplary embodiment and variations described below are only examples of the present disclosure and should not be construed as limiting. The exemplary embodiment and variations described below may be readily modified in various manners depending on a design choice or any other factor, as long as they do not deviate from the technical idea according to the present disclosure.

### Embodiment

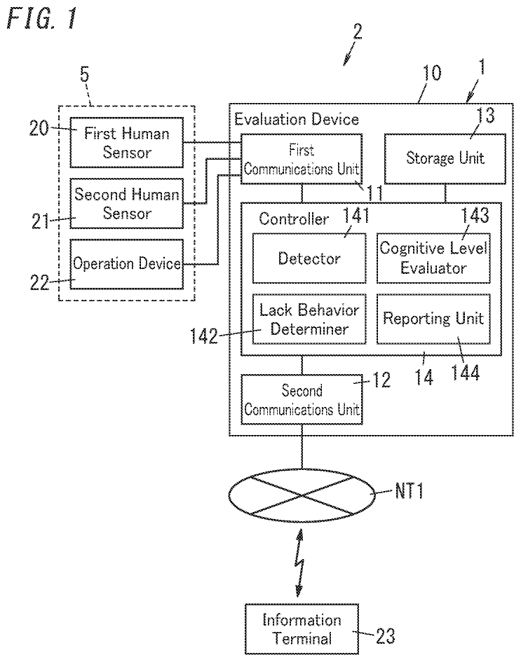

Hereinafter, a cognitive level evaluation system 1 according to an exemplary embodiment will be described with reference to FIGS. 1 to 6.

### (1) Overview

An evaluating system 2 according to the present exemplary embodiment includes an evaluation device 10 as the cognitive level evaluation system 1, a first human sensor 20, a second human sensor 21, an operation device 22, and an information terminal 23, as illustrated in FIG. 1. The information terminal 23 may be implemented as a smartphone or a tablet terminal, for example.

The evaluation device 10 is configured to communicate with the first human sensor 20, the second human sensor 21, the operation device 22, and the information terminal 23. For example, the evaluation device 10 is configured to communicate with the first human sensor 20, the second human sensor 21, and the operation device 22 by wire communication or wireless communication. The evaluation device 10 is configured to communicate with the information terminal 23 via a network NT1 such as the Internet.

The evaluation device 10 evaluates a cognitive level of a person (subject) as a target for which cognitive level is evaluated, based on a behavior of the subject in a space (toilet space) 5 where a toilet device 30 is installed. In this embodiment, the toilet device 30 is assumed to be used by only one subject in the following description. The subject is a target for which cognitive level is evaluated as described above.

In this embodiment, the cognitive level corresponds to a degree of dementia. The cognitive level may be a degree of suspicion of dementia or a probability of dementia. Alternatively, the cognitive level may be a level corresponding to a range of a mild cognitive impairment (MCI) that is milder than the dementia.

The cognitive level may also be defined in two stages: a normal level corresponding to a range of healthy persons and an abnormal level corresponding to a range of the MCI or the dementia. The abnormal level may be further subdivided into multiple stages in detail, such as the MCI, mild dementia, moderate dementia, and severe dementia.

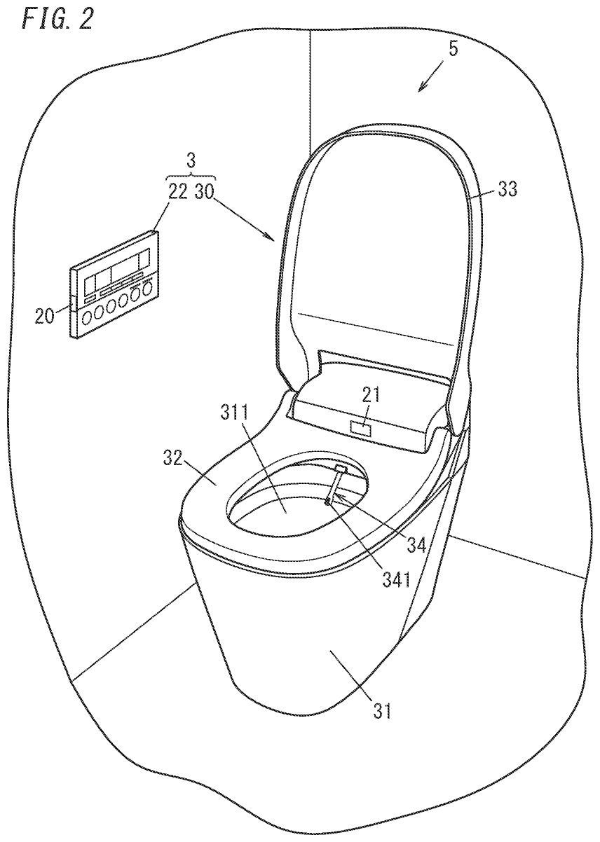

As illustrated in FIG. 2, the toilet device 30 includes a flush toilet bowl 31, a toilet seat 32, a toilet lid 33, and a washing device 34. The toilet bowl 31 is a stool type toilet bowl. The operation device 22 may be provided integrally with or separately from the toilet device 30. In the following description, the operation device 22 is assumed to be installed on the wall in the toilet space 5 so as to be provided separately from the toilet device 30 (i.e., separately from the toilet bowl 31). Hereinafter, a state where the toilet device 30 is installed in the toilet space 5 will be described. Also, hereinafter, the toilet bowl 31 is assumed to have an upper surface provided with an opening surface 311. In this embodiment, the operation device 22 and the toilet device 30 constitute a toilet system 3.

The toilet seat 32 and the toilet lid 33 are coupled with the toilet bowl 31 so as to be movable between: an opened position of rising from the upper surface of the toilet bowl 31; and a closed position of laying down along the upper surface of the toilet bowl 31. The toilet seat 32 and the toilet lid 33 may be generally coupled with the toilet bowl 31 so as to be turnable around an axis of turning. In this embodiment, the toilet seat 32 and the toilet lid 33 are coupled with the toilet bowl 31 so as not to be separated from the toilet bowl 31.

Each of the toilet seat 32 and the toilet lid 33 moves independently by receiving a driving force from a motor (not shown) in accordance with the operation input to the operation device 22. When the toilet seat 32 is in the closed position, the toilet lid 33 is movable between the opened and closed positions. When the toilet lid 33 is in the opened position, the toilet seat 32 is movable between the opened and closed positions. That is to say, the toilet device 30 has three selectable states: a state where both the toilet seat 32 and the toilet lid 33 are in the closed position; a state where the toilet seat 32 is in the closed position, but the toilet lid 33 is in the opened position; and a state where both the toilet seat 32 and the toilet lid 33 are in the opened position.

The timing of discharging wash water into the toilet bowl 31 and the amount of wash water are controlled in accordance with the operation input to the operation device 22. The washing device 34 includes a nozzle 341 for discharging water to wash a private part of a person (subject) sitting on the toilet seat 32. The nozzle 341 is switched between a state of discharging water from the nozzle 341 and a state of stopping the discharge of water in accordance with the operation input to the operation device 22. The discharge of water from the nozzle 341 can wash the private part of the person sitting on the toilet seat 32. Furthermore, the washing device 34 controls the position of the nozzle 341, the pressure of water to be ejected from the nozzle 341, the manner of ejecting water and the like in accordance with the operation input to the operation device 22.

The first human sensor 20, the second human sensor 21 and the operation device 22 are disposed together with the toilet device 30 in the toilet space 5.

The first human sensor 20 is a sensor for sensing a person (subject) entering or leaving the toilet space 5. The first human sensor 20 is configured to communicate with the evaluation device 10. The first human sensor 20 is disposed on a lateral surface of a housing of the operation device 22, for example.

The second human sensor 21 is a sensor for sensing whether or not the toilet device 30 is used. The second human sensor 21 is configured to communicate with the evaluation device 10. The second human sensor 21 is installed at the toilet bowl 31, for example. The second human sensor 21 installed at the toilet bowl 31 senses the subject standing up at the front of the toilet bowl 31 or the subject sitting on the toilet seat 32.

The second human sensor 21 has a sensing range narrower than that of the first human sensor 20. Therefore, there are three states in the toilet space 5; an occupied state where the first human sensor 20 senses the subject, but the second human sensor 21 senses no subject; a used state where both the first human sensor 20 and the second human sensor 21 sense the subject; and an absent state where both the first human sensor 20 and the second human sensor 21 sense no subject.

The occupied state means a state where the subject is present in the toilet space 5 but not using the toilet device 30. The used state means a state where the subject is present in the toilet space 5 and further using the toilet device 30. The absent state means a state where the subject is absent in the toilet space 5.

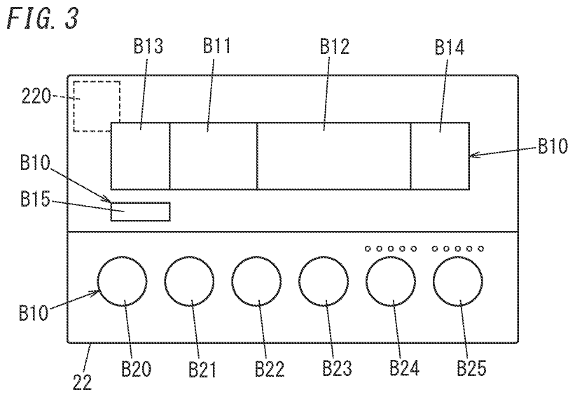

As described above, the operation device 22 is used for the operation input to the toilet device 30. The operation device 22 is configured to communicate with the evaluation device 10. The operation device 22 includes a plurality of operation parts B11, B12, B13, B14, B15, B20, B21, B22, B23, B24 and B25 (see FIG. 3). In the following description, when it is not necessary to individually distinguish between the plurality of operation parts B11, B12, B13, B14, B15, B20, B21, B22, B23, B24 and B25 of the operation device 22, each may be sometimes referred to as an operation part B10.

The operation device 22 outputs, to the evaluation device 10, operation information representing the kind of operation part B10 operated, in response to each operation input to the operation device 22.

The evaluation device 10 determines (detects) whether or not the subject is present in the toilet space 5 and whether or not the subject is using the toilet device 30 when being present in the toilet space 5, based on a sensing result of each of the first human sensor 20 and the second human sensor 21. The evaluation device 10 performs a detection relating to the operation input to the operation device 22 based on the operation information received from the operation device 22.

The evaluation device 10 evaluates the cognitive level of the subject based on the detection result, and reports an evaluation result to the information terminal 23 via the network NT1. Furthermore, the evaluation device 10 reports, when finding that lacking behavior in the toilet space 5 is present, information about the lacking behavior to the operation device 22.

When receiving the evaluation result for the subject from the evaluation device 10 via the network NT1, the information terminal 23 allows a display unit thereof to display the evaluation result received.

### (2) Configuration

(2-1) Evaluation Device

As illustrated in FIG. 1, the evaluation device 10 includes a first communications unit 11, a second communications unit 12, a storage unit 13 and a controller 14.

The evaluation device 10 may include a computer system including a processor and a memory, for example. The computer system performs the function of the controller 14 by making the processor execute a program stored in the memory. In this embodiment, the program to be executed by the processor is stored in advance in the memory of the computer system. However, this is only an example and should not be construed as limiting. The program may also be distributed after having been stored in a non-transitory storage medium such as a memory card or downloaded via a telecommunications line such as the Internet.

The first communications unit 11 has a communication interface for communicating with the first human sensor 20, the second human sensor 21 and the operation device 22.

The second communications unit 12 has a communication interface for communicating with the information terminal 23.

The storage unit 13 may include a memory device such as a hard disk drive (HDD) or a solid state drive (SSD).

The storage unit 13 stores (memorizes) a behavior pattern of the subject from when the subject begins to use the toilet device 30 until when the subject leaves the toilet space 5. For example, the storage unit 13 stores a behavior pattern including an operation of opening the toilet lid 33, an operation of opening the toilet seat 32, an operation of flushing the toilet for treating excrement, an operation of closing the toilet seat 32, and an operation of closing the toilet lid 33, where each operation is associated with a rank number of behavior (indicating where the corresponding operation is placed in an order of a plurality of operations). Also, the storage unit 13 stores a behavior pattern including an operation of opening the toilet lid 33, an operation of flushing the toilet for treating excrement, an operation of discharging water from the nozzle 341, an operation of stopping the discharge of water from the nozzle 341, and an operation of closing the toilet lid 33, where each operation is associated with a rank number of behavior (indicating where the corresponding operation is placed in the order of the plurality of operations).

The storage unit 13 further stores information about detection of the behavior of the subject in the toilet space 5, which is obtained based on the sensing result of the first human sensor 20, the sensing result of the second human sensor 21 and the detection relating to the operation input to the operation device 22. For example, the storage unit 13 stores, in time series, information representing the presence or absence of the behavior in toilet space 5 (i.e., usage information representing that the toilet device 30 is used or not), after the subject is detected to enter the toilet space 5. The storage unit 13 stores, in time series, behavior order information representing an order of the plurality of the behaviors in the toilet space 5 each time the subject is detected to enter the toilet space 5. Also, the storage unit 13 stores, in time series, staying time information representing a staying time of the subject staying in the toilet space 5 each time the subject is detected to enter the toilet space 5. Also, the storage unit 13 stores, in time series, information about whether or not the same behaviors are performed until the subject leaves the toilet space 5 (i.e., overlapping behaviors information representing the presence or absence of overlapping behaviors) each time the subject is detected to enter the toilet space 5. Also, the storage unit 13 stores, in time series, information representing whether or not the subject is lacking in a certain behavior in the toilet space 5 (i.e., lack behavior presence/absence information representing the presence or absence of lacking behavior in the toilet space 5).

As illustrated in FIG. 1, the controller 14 includes a detector 141, a lack behavior determiner 142, a cognitive level evaluator 143 and a reporting unit 144.

The detector 141 is configured to detect the behavior of the subject in the toilet space. For example, the detector 141 detects a plurality of the behaviors of the subject in the toilet space. Hereinafter, the detection to be performed by the detector 141 will be described in detail.

The detector 141 measures (detects), as the staying time, a time period from when the first human sensor 20 senses that the subject enters the toilet space 5 to when the first human sensor 20 senses that the subject leaves the toilet space 5. The detector 141 allows the storage unit 13 to store staying time information representing the staying time detected.

When the first human sensor 20 senses that the subject enters the toilet space 5, the detector 141 detects, as the order of the plurality of the behaviors, an order of a plurality of operations sequentially input to the operation device 22 by the subject before the subject leaves the toilet space 5, and allows the storage unit 13 to store the order of the plurality of the behaviors. For example, the detector 141 receives the operation information from the operation device 22 each time the operation device 22 is operated. The detector 141 detects a kind of each operation performed to the operation device 22 based on the kind of operation part B10 represented in the operation information received. The detector 141 allows the storage unit 13 to store, as behavior order information, information including: all kinds of operations detected during the subject's stay in the toilet space 5; and rank numbers of operations (each of which indicates where the corresponding operation is placed in the order of the plurality of operations) respectively associated with all kinds of operations.

The detector 141 detects whether or not all kinds of operations, detected during the subject's stay in the toilet space 5, include overlapping kinds of operations, based on the operation contents performed for the operation device 22. The detector 141 allow the storage unit 13 to store, as the overlapping behaviors information, the detection result about the presence or absence of the overlapping kinds of operations.

The detector 141 detects whether or not the toilet device 30 is used by the subject, namely the presence or absence of the behavior in toilet space 5. The detector 141 allows the storage unit 13 to store, as the usage information, the detection result about whether or not the toilet device 30 is used. For example, the detector 141 determines that the toilet device 30 is not used (i.e., no behavior is performed in the toilet space 5), if no operation of the subject is detected regardless of the sensing result of the second human sensor 21 after the first human sensor 20 senses the person. The detector 141 determines that the toilet device 30 is used (i.e., the behavior is performed in the toilet space 5), if the operation of the subject is detected regardless of the sensing result of the second human sensor 21 after the first human sensor 20 senses the person.

The lack behavior determiner 142 determines whether or not the lacking behavior is present in the toilet space 5, based on the detection result of the detector 141. More specifically, the lack behavior determiner 142 determines whether or not the lacking behavior is present in the toilet space 5 by comparing the behavior order information as the last detection result with the behavior pattern memorized in the storage unit 13. For example, the lack behavior determiner 142 determines the presence or absence of a behavior(s), which is included in the behavior pattern and not included in the behavior order information. The behavior, which is included in the behavior pattern and not included in the behavior order information, corresponds to the lacking behavior.

When determining that the lacking behavior is present in the toilet space 5, the lack behavior determiner 142 controls the reporting unit 144 to report lack behavior information relating to the lacking behavior to the operation device 22. In addition, the lack behavior determiner 142 allows the storage unit 13 to store the determination result about the presence or absence of the lacking behavior, as the lack behavior presence/absence information.

The cognitive level evaluator 143 is configured to evaluate the cognitive level of the subject based on the detection result of the detector 141.

The cognitive level evaluator 143 determines whether or not the toilet device 30 is used based on the usage information as the last detection result. When determining that the toilet device 30 is not used during the last stay in the toilet space 5, the cognitive level evaluator 143 calculates an unused rate at which the toilet device 30 is not used based on a plurality of pieces of the usage information included in a prescribed time period in the past based on the last detection result (e.g., the past one month). The cognitive level evaluator 143 evaluates the cognitive level of the subject based on the unused rate calculated. For example, when the unused rate is equal to or less than a first prescribed value, the cognitive level evaluator 143 determines that the cognitive level is at the normal level. When the unused rate is more than the first prescribed value, the cognitive level evaluator 143 determines that the cognitive level is at the abnormal level. Note that, when the abnormal level is further subdivided into multiple stages in detail, the cognitive level evaluator 143 may assign, as the abnormal level, one stage corresponding to the unused rate, of the multiple stages.

The cognitive level evaluator 143 compares the behavior order information as the last detection result with the behavior pattern memorized in the storage unit 13, and determines whether or not the order of the plurality of the behaviors included in the behavior order information matches with the behavioral order represented in the behavior pattern. When determining that the order does not match with the behavioral order, the cognitive level evaluator 143 calculates a mismatch rate at which the behavior pattern does not match with the order of the plurality of the behaviors based on a plurality of pieces of the behavior order information included in a prescribed time period in the past based on the last detection result (e.g., the past one month). The cognitive level evaluator 143 evaluates the cognitive level of the subject based on the mismatch rate calculated. For example, when the mismatch rate is equal to or less than a second prescribed value, the cognitive level evaluator 143 determines that the cognitive level is at the normal level. When the mismatch rate is more than the second prescribed value, the cognitive level evaluator 143 determines that the cognitive level is at the abnormal level. Note that, when the abnormal level is further subdivided into multiple stages in detail, the cognitive level evaluator 143 may assign, as the abnormal level, one stage corresponding to the mismatch rate, of the multiple stages.

The cognitive level evaluator 143 determines whether or not the staying time represented in the staying time information as the last detection result exceeds a prescribed time. When determining that the staying time exceeds the prescribed time, the cognitive level evaluator 143 calculates an exceeding frequency at which the staying time exceeds the prescribed time, based on a plurality of pieces of the staying time information included in a prescribed time period in the past based on the last detection result (e.g., the past one month). The cognitive level evaluator 143 evaluates the cognitive level of the subject based on the exceeding frequency calculated. For example, when the exceeding frequency is equal to or less than a third prescribed value, the cognitive level evaluator 143 determines that the cognitive level is at the normal level. When the exceeding frequency is more than the third prescribed value, the cognitive level evaluator 143 determines that the cognitive level is at the abnormal level. Note that, when the abnormal level is further subdivided into multiple stages in detail, the cognitive level evaluator 143 may assign, as the abnormal level, one stage corresponding to the exceeding frequency, of the multiple stages.

The cognitive level evaluator 143 determines the presence or absence of the overlapping behaviors based on the overlapping behaviors information as the last detection result. When determining that the overlapping behaviors are present, the cognitive level evaluator 143 calculates the number of pieces of overlapping behaviors information including the overlapping behaviors (i.e., an overlapping frequency at which the presence of the overlapping behaviors is detected), based on a plurality of pieces of the overlapping behaviors information included in a prescribed time period in the past based on the last detection result (e.g., the past one month). The cognitive level evaluator 143 evaluates the cognitive level of the subject based on the overlapping frequency calculated. For example, when the overlapping frequency is equal to or less than a fourth prescribed value, the cognitive level evaluator 143 determines that the cognitive level is at the normal level. When the overlapping frequency is more than the fourth prescribed value, the cognitive level evaluator 143 determines that the cognitive level is at the abnormal level. Note that, when the abnormal level is further subdivided into multiple stages in detail, the cognitive level evaluator 143 may assign, as the abnormal level, one stage corresponding to the overlapping frequency, of the multiple stages.

When the presence of the lacking behavior is represented in the determination result about the presence or absence of the lacking behavior by the lack behavior determiner 142 using the behavior order information as the last detection result, the cognitive level evaluator 143 calculates a lack frequency as the number of times the presence of the lacking behavior is detected, of the number of usage times of the toilet space 5 in a prescribed time period in the past based on the last detection result (e.g., the past one month). The cognitive level evaluator 143 evaluates the cognitive level of the subject based on the lack frequency calculated. For example, when the lack frequency is equal to or less than a fifth prescribed value, the cognitive level evaluator 143 determines that the cognitive level is at the normal level. When the lack frequency is more than the fifth prescribed value, the cognitive level evaluator 143 determines that the cognitive level is at the abnormal level. Note that, when the abnormal level is further subdivided into multiple stages in detail, the cognitive level evaluator 143 may assign, as the abnormal level, one stage corresponding to the lack frequency, of the multiple stages.

The cognitive level evaluator 143 transmits, as the evaluation result, the evaluated cognitive level to the information terminal 23 via the network NT1. The information terminal 23 allows its display unit to display the evaluation result (cognitive level) received.

The reporting unit 144, when the presence of the lacking behavior is detected in the toilet space 5, reports the lack behavior information relating to the lacking behavior. More specifically, the reporting unit 144 transmits, as the lack behavior information, voice data in accordance with the lacking behavior to the operation device 22 via the first communications unit 11. For example, when the lacking behavior is the operation of closing the toilet lid 33, the reporting unit 144 outputs, as the lack behavior information, the voice data relating to a voice for prompting closing the toilet lid 33 to the operation device 22.

(2-2) Operation Device

FIG. 3 shows one example of the operation device 22. The operation device 22 in FIG. 3 includes a plurality of the operation parts B10. The number of the operation parts B10 shown in FIG. 3 is eleven. However, the number of the operation parts B10 provided for the operation device 22 may be modified as appropriate depending on a design choice or any other factor.

More specifically, the operation device 22 includes four operation parts B11, B12, B13 and B14 arranged in an upper region, and six operation parts B20, B21, B22, B23, B24 and B25 arranged in a lower region. The operation device 22 further includes one operation part B15 disposed (in a middle region) between the upper region and the lower region.

All the operation parts B11, B12, B13 and B14, the operation parts B20, B21, B22, B23, B24 and B25, and the operation part B15 are push buttons. Each of the operation parts B24 and B25 may be implemented as a single push button with two operational positions. That is to say, the single push button of each of the operation parts B24 and B25 practically functions as two push buttons.

The operation parts B11 and B12 are designed to select the amount of water to be used for flushing the toilet bowl 31 from two stages. That is to say, when any one of the operation parts B11 and B12 is operated, the toilet bowl 31 is flushed at the amount of water associated with the operation part operated. The operation part B13 is a button for controlling opening or closing of the toilet seat 32. The toilet seat 32 is moved from the closed state to the opened state, and vice versa in response to each operation to the operation part B13. The operation part B14 is a button for controlling opening or closing of the toilet lid 33. The toilet lid 33 is moved from the closed state to the opened state, and vice versa in response to each operation to the operation part B14.

The operation parts B21, B22 and B23 arranged in the lower region are operated for selecting the types of operation modes of the washing device 34. The operation part B20 is operated for stopping the operation of the washing device 34. The operation part B24 is operated for adjusting the pressure of water to be ejected from the nozzle 341. The operation part B24 is designed to accept either an operation of increasing the pressure or an operation of decreasing the pressure in accordance with the region of the operation part B24 operated. The operation part B25 is operated for adjusting an ejection position of water to be ejected from the nozzle 341. The position of water to be ejected from the nozzle 341 can be adjusted on the straight line. The operation part B25 is designed to accept either an operation of changing the ejection position in one direction or an operation of changing the ejection position in the other direction in accordance with the region of the operation part B25 operated. Note that, the operation part B24 is a button for stepwise adjusting the pressure of water. The operation part B25 is a button for stepwise adjusting the ejection position of water. The operation part B15 disposed in the middle region is operated for generating pseudo flowing water sound or the like.

The operation device 22 outputs, to the evaluation device 10, the operation information representing the kind of the operation part B10 operated in response to each operation input to the operation device 22.

The operation device 22 further includes a loudspeaker 220. The operation device 22 outputs the voice corresponding to the voice data received from the evaluation device 10. For example, when receiving, as the lack behavior information, the voice data for prompting the operation of closing the toilet lid 33 from the evaluation device 10, the operation device 22 allows the loudspeaker 220 to output the voice message (e.g., “Please close the toilet lid”) corresponding to the voice data received.

### (3) Operation

(3-1) Overall Operation

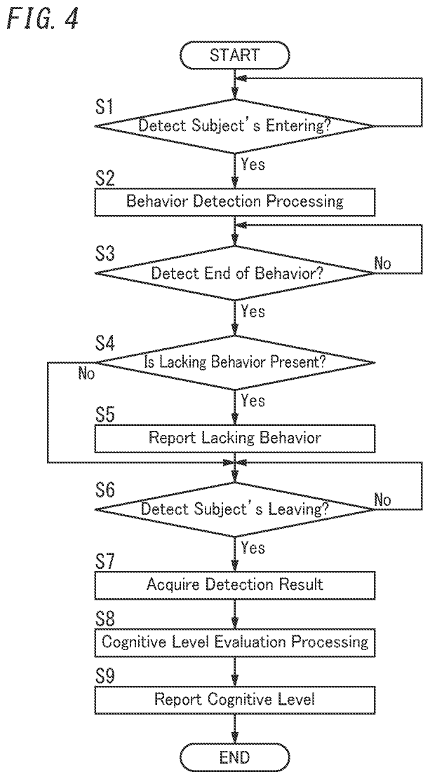

Herein, the overall operation relating to the evaluation of the cognitive level to be performed by the evaluation device 10 will be described with reference to FIG. 4.

The detector 141 determines whether or not the first human sensor 20 senses that the subject enters the toilet space 5 (in a step S1). More specifically, the detector 141 determines whether or not the sensing result of the first human sensor 20 represents that a person is sensed in the toilet space 5.

If determining that the first human sensor 20 senses that no subject enters the toilet space 5 (if the answer is “No” in the step S1), the detector 141 repeats the operation for the step S1. That is to say, if determining that the sensing result of the first human sensor 20 represents that no person is sensed in the toilet space 5, the detector 141 repeats the step S1.

If determining that the first human sensor 20 senses that the subject enters the toilet space 5 (if the answer is “Yes” in the step S1), the detector 141 performs the behavior detection processing (in a step S2). That is to say, if determining that the sensing result of the first human sensor 20 represents that a person is sensed in the toilet space 5, the detector 141 performs the behavior detection processing.

For example, the detector 141 starts measuring the staying time to detect the staying time until the subject leaves the toilet space 5. Also, the detector 141 receives the operation information from the operation device 22 in response to each operation input to the operation device 22. That is to say, the detector 141 sequentially receives one or more pieces of the operation information during the time period from when the subject enters the toilet space 5 to when the subject leaves the toilet space 5. The detector 141 identifies the kind of the operation part B10 corresponding to each piece of the operation information in response to the reception of the corresponding piece of the operation information. The detector 141 identifies (detects): a kind of operation corresponding to the kind of the operation part B10 identified; and a rank number indicating where the corresponding piece of operation information is placed in order of the one or more pieces of the operation information received so far.

The detector 141 determines whether or not the subject ends behaving in the toilet space 5 (in a step S3). More specifically, the detector 141 determines whether or not the second human sensor 21 senses the subject. That is to say, the detector 141 determines whether or not the sensing result of the second human sensor 21 represents that a person is sensed in the toilet space 5.

If determining that the subject has not yet ended behaving in the toilet space 5 (if the answer is “No” in the step S3), the detector 141 continues the behavior detection processing. Note that, the behavior detection processing is continued until a step S8 described later is started.

If the detector 141 determines that the subject has ended behaving in the toilet space 5 (if the answer is “Yes” in the step S3), the lack behavior determiner 142 determines the presence or absence of the lacking behavior in the toilet space 5 based the detection result of the detector 141 (in a step S4). The lack behavior determiner 142 allows the storage unit 13 to store the determination result about the presence or absence of the lacking behavior, as the lack behavior presence/absence information.

If the lack behavior determiner 142 determines that the lacking behavior is present in the toilet space 5 (if the answer is “Yes” in the step S4), the reporting unit 144 reports the lack behavior information relating to the lacking behavior to the operation device 22 (in a step S5).

If the lack behavior determiner 142 determines that the lacking behavior is absent in the toilet space 5 (if the answer is “No” in the step S4), or after the reporting unit 144 transmits the lack behavior information to the operation device 22 in the step S5, the detector 141 detects whether or not the subject leaves the toilet space 5 (in a step S6). More specifically, the detector 141 determines whether or not the sensing result of the first human sensor 20 represents that no person is sensed in the toilet space 5.

If determining that the subject has not yet left the toilet space 5 (if the answer is “No” in the step S6), the detector 141 repeats the operation for the step S6. That is to say, if determining that the sensing result of the first human sensor 20 does not represent that no person is sensed in the toilet space 5 (in other words, if determining that the sensing result of the first human sensor 20 represents that a person is sensed in the toilet space 5), the detector 141 repeats the step S6.

If determining that the subject has left the toilet space 5 (if the answer is “Yes” in the step S6), that is to say, if determining that the sensing result of the first human sensor 20 represents that no person is sensed in the toilet space 5, the detector 141 acquires the detection result in the behavior detection processing (in a step S7).

More specifically, the detector 141 stops measuring the staying time to acquire the staying time from when the subject enters the toilet space 5 to when the subject leaves the toilet space 5. The detector 141 allows the storage unit 13 to store the staying time information representing the staying time acquired. The detector 141 acquires (generates) the behavior order information including: one or more kinds of operations detected in the behavior detection processing; and the rank numbers of operations (each of which indicates where the corresponding operation is placed in the order of the plurality of operations) respectively associated with the one or more kinds of operations, and allows the storage unit 13 to store the behavior order information.

In addition, the detector 141 detects (acquires) the information about the presence or absence of the overlapping kinds of operations of all operations detected during staying, based on the operation contents performed with respect to the operation device 22. The detector 141 allows the storage unit 13 to store the detection result about the presence or absence of the overlapping kinds of operations, as the overlapping behaviors information.

Also, the detector 141 detects (acquires) the information about whether or not the toilet device 30 is used by the subject (i.e., the presence or absence of the behavior in the toilet space 5). The detector 141 detects the information about whether or not the toilet device 30 is used (i.e., the presence or absence of the behavior in the toilet space 5), based on the sensing result of the second human sensor 21 and the detection of the operations input to the operation device 22 during the time period from when the subject enters the toilet space 5 to when the subject leaves the toilet space 5. For example, when receiving the sensing result representing that a person is sensed from the second human sensor 21, the detector 141 decides that the subject sits on the toilet seat 32, namely that the toilet device 30 is used. Also, when receiving the operation information from the operation device 22, the detector 141 detects that the toilet device 30 is used. The detector 141 allows the storage unit 13 to store the detection result about whether or not the toilet device 30 is used, as the usage information.

The controller 14 performs the cognitive level evaluation processing to evaluate the subject's cognitive level (in a step S8). The details of the cognitive level evaluation processing will be described later.

The cognitive level evaluator 143 reports the evaluated cognitive level as the evaluation result to the information terminal 23 (in a step S9). In this case, the information terminal 23 allows its display unit to display the evaluation result (cognitive level) received.

(3-2) Cognitive Level Evaluation Processing

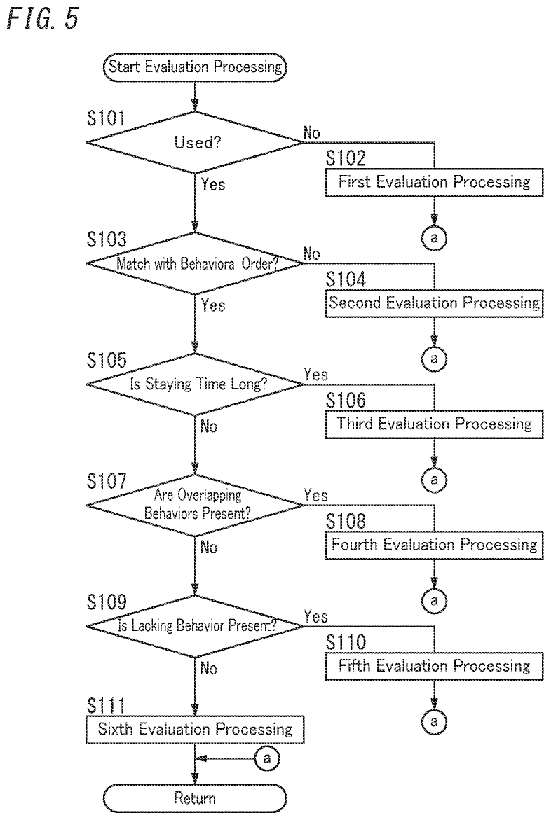

Herein, the details of the cognitive level evaluation processing will be described with reference to FIG. 5.

The cognitive level evaluator 143 determines whether or not the toilet device 30 is used based on the usage information as the last detection result (in a step S101).

If determining that the toilet device 30 is not used during the last staying in the toilet space 5 (if the answer is “No” in the step S101), the cognitive level evaluator 143 performs a first evaluation processing (in a step S102). More specifically, the cognitive level evaluator 143 calculates the unused rate in the prescribed time period in the past based on the last detection result. The cognitive level evaluator 143 evaluates the subject's cognitive level based on the unused rate calculated.

If determining that the toilet device 30 is used during the last staying in the toilet space 5 (if the answer is “Yes” in the step S101), the cognitive level evaluator 143 determines whether or not the order of the plurality of the behaviors included in the behavior order information as the last detection result matches with the behavioral order represented in the behavior pattern (in a step S103).

If determining that the order does not match with the behavioral order (if the answer is “No” in the step S103), the cognitive level evaluator 143 performs a second evaluation processing (in a step S104). More specifically, the cognitive level evaluator 143 calculates the mismatch rate in the prescribed time period in the past based on the last detection result. The cognitive level evaluator 143 evaluates the subject's cognitive level based on the mismatch rate calculated.

If determining that the order matches with the behavioral order (if the answer is “Yes” in the step S103), the cognitive level evaluator 143 determines whether or not the staying time is long, which is represented in the staying time information as the last detection result (in a step S105). More specifically, the cognitive level evaluator 143 determines whether or not the staying time exceeds the prescribed time.

If determining that the staying time is long, that is to say, the staying time exceeds the prescribed time (if the answer is “Yes” in the step S105), the cognitive level evaluator 143 performs a third evaluation processing (in a step S106). More specifically, the cognitive level evaluator 143 calculates the exceeding frequency in the prescribed time period in the past based on the last detection result. The cognitive level evaluator 143 evaluates the subject's cognitive level based on the exceeding frequency calculated.

If determining that the staying time is not long, that is to say, the staying time does not exceed the prescribed time (if the answer is “No” in the step S105), the cognitive level evaluator 143 determines whether or not the overlapping behaviors are present based on the overlapping behaviors information as the last detection result (in a step S107).

If determining that the overlapping behaviors are present (if the answer is “Yes” in the step S107), the cognitive level evaluator 143 performs a fourth evaluation processing (in a step S108). More specifically, the cognitive level evaluator 143 calculates the overlapping frequency in the prescribed time period in the past based on the last detection result. The cognitive level evaluator 143 evaluates the subject's cognitive level based on the overlapping frequency calculated.

If determining that the overlapping behaviors are not present (if the answer is “No” in the step S107), the cognitive level evaluator 143 determines whether or not the lacking behavior is present based on the determination result of the lack behavior determiner 142 (in a step S109).

If determining that the lacking behavior is present (if the answer is “Yes” in the step S109), the cognitive level evaluator 143 performs a fifth evaluation processing (in a step S110). More specifically, the cognitive level evaluator 143 calculates the lack frequency in the prescribed time period in the past based on the last detection result. The cognitive level evaluator 143 evaluates the subject's cognitive level based on the lack frequency calculated.

If determining that the lacking behavior is not present (if the answer is “No” in the step S109), the cognitive level evaluator 143 performs a sixth evaluation processing (in a step S111). More specifically, the cognitive level evaluator 143 evaluates that the cognitive level is at the normal level.

### (4) Specific Example

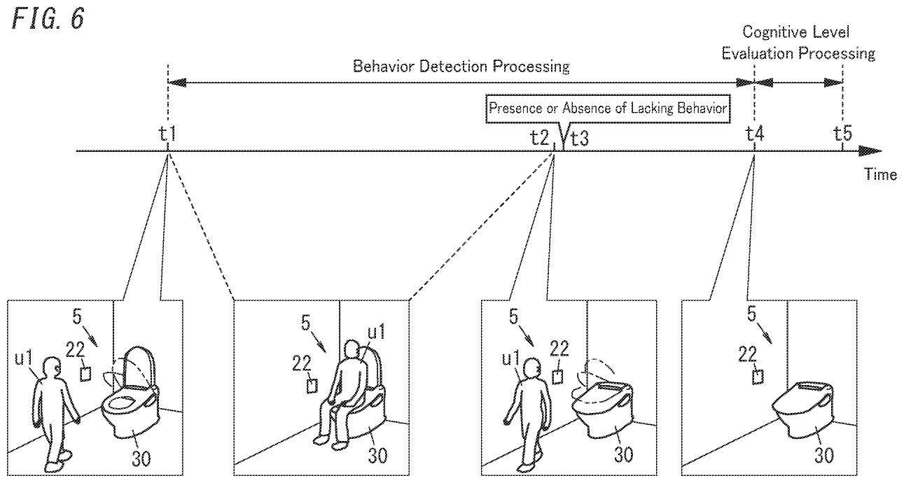

Herein, the operation about how the evaluation device 10 operates will be described with reference to FIG. 6, using the specific example that the subject uses the toilet space 5.

When a subject u1 enters the toilet space 5, the first human sensor 20 senses the person (at a time t1). At that time, the detector 141 detests that the subject u1 enters the toilet space 5 based on the sensing result of the first human sensor 20, and starts the behavior detection processing. The detector 141 performs, for example, measuring of the staying time and identifying of the kinds of operations and the rank numbers of operations, as the behavior detection processing.

After entering the toilet space 5, the subject u1 sits on the toilet seat 32 of the toilet device 30 and then separates from the toilet device 30 (from the time t1 to a time t2). When the operation device 22 is operated between the times t1 and t2, the detector 141 identifies the kinds of operations and the rank numbers of operations.

When the subject u1 separates from the toilet device 30, the first human sensor 20 senses the person but the second human sensor 21 senses no person. At that time, the detector 141 detects that the subject u1 has ended using the toilet device 30, based on the sensing result of the second human sensor 21, and the lack behavior determiner 142 determines whether or not the lacking behavior is present (at a time t3). When the lack behavior determiner 142 determines that the lacking behavior is present, the reporting unit 144 reports the lack behavior information relating to the lacking behavior. Note that, even when the second human sensor 21 senses no person, the behavior detection processing is continuously performed while the first human sensor 20 senses the person.

When the subject u1 separates from the toilet device 30 and then leaves the toilet space 5, the first human sensor 20 results in sensing no person (at a time t4). In other words, when the subject u1 leaves the toilet space 5, the first human sensor 20 results in sensing that the person is absent. At that time, the detector 141 detects that the subject u1 has left the toilet space 5 based on the sensing result of the first human sensor 20, and performs the cognitive level evaluation processing for the subject. The detector 141 reports the evaluation result to the information terminal 23 (at a time t5).

### (5) Advantage

As described above, the cognitive level evaluation system 1 (evaluation device 10) according to this embodiment includes the detector 141 and the cognitive level evaluator 143. The detector 141 detects the subject's behavior in the toilet space 5 based on the sensing result of the first human sensor 20, the sensing result of the second human sensor 21 and the detection result of the operation input to the operation device 22. For example, the detector 141 detects, as the subject's behavior, the staying time of the subject in the toilet space 5, whether or not the operation device 22 is used, the order of the plurality of operations, the presence or absence of the overlapping behaviors, and the presence or absence of the lacking behavior. The cognitive level evaluator 143 evaluates the cognitive level of the subject based on the behavior detected.

According to this configuration, the cognitive level evaluation system 1 detects the subject's behavior in the toilet space 5, as one aspect of life, and evaluates the cognitive level. Therefore, the cognitive level evaluation system 1 can realize evaluating the cognitive level of the subject based on the behavior in one aspect of life.

Note that, the configuration is optional, where the detector 141 detect, as the subject's behavior, all of the staying time of the subject in the toilet space 5, whether or not the operation device 22 is used, the order of the plurality of operations, the presence or absence of the overlapping behaviors, and the presence or absence of the lacking behavior. The detector 141 may be configured to detect at least one of the staying time of the subject in the toilet space 5, whether or not the operation device 22 is used, the order of the plurality of operations, the presence or absence of the overlapping behaviors, or the presence or absence of the lacking behavior.

### (6) Variations

Hereinafter, some exemplary variations will be listed. Note that, each of the variations in the following description may be adopted in combination with the exemplary embodiment described above as appropriate.

(6-1) First Variation

The detector 141 may measure a time required for the behavior detected. For example, when the subject performs a behavior for washing a private part of himself/herself, the detector 141 may measure a time required for the behavior.

For example, the detector 141 measures, as the time required for the behavior for washing the private part, a time between a time point at which an operation for directing starting the washing is performed and a time point at which an operation for directing ending the washing is performed.

In this case, the cognitive level evaluator 143 evaluates the cognitive level of the subject based on the time measured. For example, when the measured time is equal to or less than a first prescribed time, or equal to or more than a second prescribed time, the cognitive level evaluator 143 evaluates that the cognitive level is at the abnormal level. When the measured time exceeds the first prescribed time but is less than the second prescribed time, the cognitive level evaluator 143 evaluates that the cognitive level is at the normal level. Note that, when the abnormal level is further subdivided into multiple stages in detail, the cognitive level evaluator 143 may assign, as the abnormal level, one stage corresponding to the exceeding frequency, of the multiple stages.

(6-2) Second Variation

The detector 141 may detect the kind of the behavior of the subject in the toilet space 5. In this case, the cognitive level evaluator 143 may evaluate the cognitive level of the subject based on the kind of the behavior detected.

For example, the detector 141 may detect, as the kind of the behavior, the amount of toilet paper consumed by the subject. When the toilet paper is a roll type, the detector 141 can detect the amount of toilet paper consumed, based on a sensing result about a rotation number of the toilet paper sensed by a gyro sensor of sensing rotation of the toilet paper.

The cognitive level evaluator 143 evaluates the cognitive level of the subject based on the amount of toilet paper consumed. For example, when the amount of toilet paper consumed is equal to or less than a first prescribed amount, the cognitive level evaluator 143 evaluates that the cognitive level is at the abnormal level. When the amount of toilet paper consumed is more than the first prescribed amount, the cognitive level evaluator 143 evaluates that the cognitive level is at the normal level. Note that, when the abnormal level is further subdivided into multiple stages in detail, the cognitive level evaluator 143 may assign, as the abnormal level, one stage corresponding to the amount of toilet paper consumed, of the multiple stages.

(6-3) Third Variation

The cognitive level evaluator 143 may evaluate the cognitive level of the subject based on the operation input to the operation part(s) B10. For example, the cognitive level evaluator 143 may evaluate the cognitive level of the subject based on a rate of long pressing operation to a push button(s) as the operation part(s) B10.

In this case, the operation device 22 outputs, to the evaluation device 10, the operation information including a time over which the corresponding operation part B10 is pressed in response to each operation to the operation device 22. The detector 141 detects (acquires) the rate of long pressing operation with respect to the operation device 22. The rate of long pressing operation means a rate of the number of times of long pressing with respect to the total number of times of pressing. The total number of times of pressing means the number of times that the operation part B10 (push button) of the operation device 22 is pressed. The number of times of long pressing means the number of times that the operation part B10 of the operation device 22 is kept pressed over a time longer than a prescribed reference time. The reference time is two seconds, for example. The reference time may be a fixed time, or a time variably defined.

Also, for example, the detector 141 may acquire the total number of times of pressing, or the number of times of long pressing. The detector 141 may acquire the rate of the long pressing operation by calculating the rate of the long pressing operation based on the total number of times of pressing and the number of times of long pressing.

The cognitive level evaluator 143 evaluates the cognitive level of the subject based on the rate of the long pressing operation acquired by the detector 141. More specifically, the cognitive level evaluator 143 relatively evaluates the cognitive level such that the lower the rate of the long pressing operation is, more severe the cognitive level is. For example, when the rate of the long pressing operation is at a first rate, the cognitive level evaluator 143 evaluates that the cognitive level is at the normal level. When the rate of the long pressing operation is at a second rate lower than the first rate, the cognitive level evaluator 143 evaluates that the cognitive level is at the abnormal level. Note that, when the abnormal level is further subdivided into multiple stages in detail, the cognitive level evaluator 143 may assign, as the abnormal level, one stage corresponding to the rate of the long pressing operation, of the multiple stages.

Alternatively, the cognitive level evaluator 143 is not limited to be configured to evaluate the cognitive level to always follow the definition that the lower the rate of the long pressing operation is, more severe the cognitive level is. The cognitive level evaluator 143 may be configured to exceptionally evaluate the cognitive level to be out of the definition that the lower the rate of the long pressing operation is, more severe the cognitive level is, in accordance with another condition or the like. Also, for example, when the rate of the long pressing operation is within a prescribed range, the cognitive level evaluator 143 may evaluate that the cognitive level is at the same level.

(6-4) Fourth Variation

As another example that the cognitive level of the subject is evaluated based on the operation to the operation part(s) B10, the cognitive level evaluator 143 may evaluate the cognitive level of the subject based on consecutive operation to the operation part(s) B10.

The detector 141 acquires (detects), as information about the consecutive operation to the operation device 22, a rate of consecutive pressing operation to the operation device 22. The rate of consecutive pressing operation means a rate of the number of times of consecutive pressing with respect to the total number of times of pressing. The total number of times of pressing means the number of times that the operation part B10 (push button) of the operation device 22 is pressed. The number of times of consecutive pressing means the number of times that the operation part B10 of the operation device 22 is pressed at a time interval shorter than a prescribed reference time interval. The reference time interval is three seconds, for example. The reference time interval may be empirically or experimentally defined as appropriate. The reference time interval may be a fixed time interval, or a time interval variably defined.

Also, for example, the detector 141 may acquire the total number of times of pressing, or the number of times of consecutive pressing. The detector 141 may acquire the rate of the consecutive pressing operation by calculating the rate of the consecutive pressing operation based on the total number of times of pressing and the number of times of consecutive pressing.

The cognitive level evaluator 143 evaluates the cognitive level based on the rate of the consecutive pressing operation acquired by the detector 141.

More specifically, the cognitive level evaluator 143 relatively evaluates the cognitive level such that the higher the rate of the consecutive pressing operation is, more severe the cognitive level is. For example, when the rate of the consecutive pressing operation is at a third rate, the cognitive level evaluator 143 evaluates that the cognitive level is at the normal level. When the rate of the consecutive pressing operation is at a fourth rate higher than the third rate, the cognitive level evaluator 143 evaluates that the cognitive level is at the abnormal level. Note that, when the abnormal level is further subdivided into multiple stages in detail, the cognitive level evaluator 143 may assign, as the abnormal level, one stage corresponding to the rate of the consecutive pressing operation, of the multiple stages.

Alternatively, the cognitive level evaluator 143 is not limited to be configured to evaluate the cognitive level to always follow the definition that the higher the rate of the consecutive pressing operation is, more severe the cognitive level is. The cognitive level evaluator 143 may be configured to exceptionally evaluate the cognitive level to be out of the definition that the higher the rate of the consecutive pressing operation is, more severe the cognitive level is, in accordance with another condition or the like. Also, for example, when the rate of the consecutive pressing operation is within a prescribed range, the cognitive level evaluator 143 may evaluate that the cognitive level is at the same level.

(6-5) Fifth Variation

The detector 141 may acquire (detect) a time of day when the behavior of the subject is detected in the toilet space 5. In this case, the cognitive level evaluator 143 evaluates the cognitive level of the subject based on the time of day when the detector 141 detects the behavior of the subject in the toilet space 5.

For example, the detector 141 detects, as the behavior, the subject entering the toilet space 5 (or leaving the toilet space 5, or both entering and leaving). The detector 141 calculates a rate of the time of day, when the behavior is detected, corresponding to a time at midnight (e.g., 0:00 AM to 05:00 AM) in a prescribed time period. When the calculated rate is equal to or less than a prescribed value, the cognitive level evaluator 143 evaluates that the cognitive level is at the normal level. When the calculated rate is more than the prescribed value, the cognitive level evaluator 143 evaluates that the cognitive level is at the abnormal level. Note that, when the abnormal level is further subdivided into multiple stages in detail, the cognitive level evaluator 143 may assign, as the abnormal level, one stage corresponding to the calculated rate, of the multiple stages.

As another example, the detector 141 may acquire a detecting frequency (i.e., the number of detection times) at which the behavior of the subject is detected in the toilet space 5 for a prescribed time period (e.g., one day). In this case, the cognitive level evaluator 143 evaluates the cognitive level of the subject based on the detecting frequency by the detector 141.

For example, the detector 141 acquires (detects), as the behavior of the subject in the toilet space 5 for one day, the number of detection times that the subject entering the toilet space 5 (or leaving the toilet space 5, or both entering and leaving) is detected. When the number of detection times acquired is equal to or less than a prescribed value, the cognitive level evaluator 143 evaluates that the cognitive level is at the normal level. When the number of detection times acquired is more than the prescribed value, the cognitive level evaluator 143 evaluates that the cognitive level is at the abnormal level. Note that, when the abnormal level is further subdivided into multiple stages in detail, the cognitive level evaluator 143 may assign, as the abnormal level, one stage corresponding to the number of detection times acquired, of the multiple stages.

As yet another example, the detector 141 may acquire both the time of day when the behavior of the subject is detected in the toilet space 5 and the detecting frequency at which the behavior of the subject is detected in the toilet space 5 for a prescribed time period (e.g., one day). In this case, the cognitive level evaluator 143 may evaluate the cognitive level of the subject based on both the time of day when the behavior of the subject is detected in the toilet space 5 and the detecting frequency at which the behavior of the subject is detected in the toilet space 5 for the prescribed time period (e.g., one day).

That is to say, the detector 141 may acquire at least one of the time of day when the behavior of the subject is detected in the toilet space 5 or the detecting frequency at which the behavior of the subject is detected in the toilet space 5 for the prescribed time period (e.g., one day). In this case, the cognitive level evaluator 143 may evaluate the cognitive level of the subject based on at least one of the time of day when the behavior of the subject is detected in the toilet space 5 or the detecting frequency at which the behavior of the subject is detected in the toilet space 5 for the prescribed time period (e.g., one day).

(6-6) Sixth Variation

The detector 141 may detect, as the behavior of the subject in the toilet space 5, an excretion action of the subject. For example, the detector 141 may detect presence or absence of excrement of the subject in the toilet bowl 31. In this case, the detector 141 detects water quality in the toilet bowl 31. When finding that the water quality upon the operation for washing the toilet bowl 31 is different from that before the subject enters the toilet space 5, the detector 141 detects that the excrement of the subject is present in the toilet bowl 31.

The cognitive level evaluator 143 evaluates the cognitive level based on the presence or absence of the excrement of the subject in the toilet bowl 31. When the detector 141 detects that the excrement of the subject is present in the toilet bowl 31, the cognitive level evaluator 143 evaluates that the cognitive level is at the normal level. When the detector 141 detects that the excrement of the subject is not present in the toilet bowl 31, the cognitive level evaluator 143 evaluates that the cognitive level is at the abnormal level.

(6-7) Seventh Variation

The operation device 22 in the exemplary embodiment described above is configured to output, to the evaluation device 10, the operation information representing the kind of the operation part B10 operated. However, such the configuration is only an example and should not be construed as limiting.

The operation device 22 may output, to the evaluation device 10, information representing the kind of the operation corresponding to the operation part B10 operated.

In this case, the detector 141 receives the information representing the kind of the operation in response to each operation input to the operation device 22. When receiving the information representing the kind of the operation, the detector 141 identifies (detects) a rank number indicating where the received information is placed in order of one or more pieces of the information received so far. After detecting that the subject leaves the toilet space 5, the detector 141 acquires (generates) the behavior order information including: one or more kinds of operations received during the subject's stay in the toilet space 5 (i.e., during use of the toilet space 5); and the rank numbers of operations (each of which indicates where the corresponding kind of operation is placed in the order of the one or more kinds of operations received so far) respectively associated with the one or more kinds of operations, and allows the storage unit 13 to store the behavior order information.

(6-8) Eighth Variation

In the exemplary embodiment described above, the toilet device 30 is assumed to be used by only one subject, but may be used by two or more subjects.

In this case, the storage unit 13 stores the detection result about the behavior of each subject in the toilet space 5. For example, each of the two or more subjects may carry a tag having an identifier for identifying the corresponding subject and capable of communicating with device. When each subject enters the toilet space 5, the evaluation device 10 acquires the identifier transmitted from the tag carried by the corresponding subject.

The evaluation device 10 evaluates the cognitive level of the subject associated with the identifier acquired. That is to say, the evaluation device 10 evaluates the cognitive level, using the detection result, memorized in the storage unit 13, corresponding to the subject associated with the identifier acquired.

Note that, the tag is used for identifying each subject in the above configuration. However, such the configuration is only an example and should not be construed as limiting. For example, a body weight sensor may be provided for the toilet seat 32, and each subject may be identified based on the body weight of the corresponding subject sit on the toilet seat 32. In short, the configuration of the evaluating system 2 is not limited as long as having a function of identifying each subject.

(6-9) Ninth Variation

The detector 141 may detect whether or not the toilet paper is used. For example, when the toilet paper is a roll type, the detector 141 may detect whether or not the toilet paper is used, based on a sensing result of a gyro sensor of sensing rotation of the toilet paper.

(6-10) Tenth Variation

In the exemplary embodiment described above, the cognitive level evaluator 143 is configured to evaluate the cognitive level of the subject, individually using each of the presence or absence of the behavior detection, the order of the plurality of the behaviors, the staying time, the presence or absence of the overlapping behaviors, and the presence or absence of the lacking behavior, as evaluation conditions. Furthermore, the cognitive level evaluator 143 is configured to evaluate the cognitive level of the subject, using each of: the time required for the behavior detected in the first variation; the kind of the behavior detected in the second variation; the operation (the rate of long pressing operation, or the consecutive pressing operation) to the operation part B10 in the third and fourth variations; the time of day when the behavior is detected in the fifth variation; and the presence or absence of the excrement in the sixth variation, as evaluation conditions. However, those configurations of the cognitive level evaluator 143 are only some examples and should not be construed as limiting.

The cognitive level evaluator 143 may evaluate the cognitive level of the subject in combination of at least two of those evaluation conditions.

(6-11) Eleventh Variation

In the exemplary embodiment described above, the cognitive level evaluator 143 is configured to evaluate the cognitive level of the subject, using the detection results in the prescribed time period. However, such the configuration is only an example and should not be construed as limiting.

The cognitive level evaluator 143 may be configured to evaluate the cognitive level of the subject, using only the last detection result.

(6-12) Other Variations

The exemplary embodiment described above is merely one example of various embodiments of the present disclosure. The exemplary embodiment may be readily modified in various manners depending on a design choice or any other factor, as long as the purpose of the present disclosure can be attained. The functions similar to the cognitive level evaluation system 1 may also be implemented as, for example, a cognitive level evaluation method, a computer program, or a non-transitory storage medium that stores the computer program. A cognitive level evaluation method according to one aspect includes a detecting step and a cognitive level evaluating step. The detecting step includes detecting a behavior of a subject in a toilet space. The cognitive level evaluating step includes evaluating a cognitive level of the subject based on a detection result in the detecting step. A (computer) program according to one aspect is designed to cause a computer system to function as the cognitive level evaluation method described above.

The cognitive level evaluation system 1 or an execution object of the cognitive level evaluation method according to the present disclosure includes a computer system. The computer system may include a processor and a memory as principal hardware components. The functions of the cognitive level evaluation system 1 or the execution object of the cognitive level evaluation method according to the present disclosure may be performed by making the processor execute a program stored in the memory of the computer system. The program may be stored in advance in the memory of the computer system. Alternatively, the program may also be downloaded through a telecommunications line or be distributed after having been recorded in some non-transitory storage medium such as a memory card, an optical disc, or a hard disk drive, any of which is readable for the computer system. The processor of the computer system may be implemented as a single or a plurality of electronic circuits including a semiconductor integrated circuit (IC) or a large-scale integrated circuit (LSI). Those electronic circuits may be either integrated together on a single chip or distributed on multiple chips, whichever is appropriate. Those multiple chips may be integrated together in a single device or distributed in multiple devices without limitation.

Also, the cognitive level evaluation system 1 as the computer system may be a system configured by one or more computers. For example, at least some functions of the cognitive level evaluation system 1 may be implemented as a cloud computing system as well.

Also, in the exemplary embodiment described above, the evaluation device 10 is provided separately from the operation device 22. However, such the configuration is only an example and should not be construed as limiting. The evaluation device 10 and the operation device 22 may be integrated together in a single housing.

(Recapitulation)

As can be seen from the foregoing description, a cognitive level evaluation system (1) according to a first aspect includes a detector (141) and a cognitive level evaluator (143). The detector (141) is configured to detect a behavior of a subject (u1) in a toilet space (5). The cognitive level evaluator (143) is configured to evaluate a cognitive level of the subject (u1) based on a detection result of the detector (141).

This configuration can realize evaluating the cognitive level of the subject based on the subject's behavior in the toilet space (5), as one aspect of life. In addition, the cognitive level of the subject can be evaluated without the need for detailed examination.

In a cognitive level evaluation system (1) according to a second aspect, which may be implemented in conjunction with the first aspect, the detector (141) is configured to detect a plurality of the behaviors of the subject (u1) in the toilet space (5). The cognitive level evaluator (143) is configured to evaluate the cognitive level of the subject based on an order of the plurality of the behaviors.

This configuration can realize evaluating the cognitive level based on the order of the plurality of the behaviors of the subject in the toilet space 5.

In a cognitive level evaluation system (1) according to a third aspect, which may be implemented in conjunction with the second aspect, the cognitive level evaluator (143) is configured to evaluate the cognitive level of the subject (u1) by comparing the order of the plurality of the behaviors detected by the detector (141) with a behavioral order previously memorized, of the subject in the toilet space (5).

This configuration can realize relatively easily evaluating the cognitive level in comparison with the behavioral order previously memorized.

In a cognitive level evaluation system (1) according to a fourth aspect, which may be implemented in conjunction with any the first aspect, the cognitive level evaluator (143) is configured to evaluate the cognitive level of the subject (u1) based on whether or not the behavior of the subject (u1) is detected in the toilet space (5) by the detector (141).

According to this configuration, the cognitive level is evaluated based on whether or not the behavior is detected. When there is a possibility that the subject has the dementia or the MCI, the subject may leave the toilet space (5) without doing anything, even when entering the toilet space (5). Thus, the cognitive level can be relatively easily evaluated by using the result about whether or not the behavior is detected in the toilet space (5).

In a cognitive level evaluation system (1) according to a fifth aspect, which may be implemented in conjunction with the first aspect, the detector (141) is configured to detect a staying time of the subject (u1) staying in the toilet space (5), as the behavior of the subject (u1) in the toilet space (5). The cognitive level evaluator (143) is configured to evaluate the cognitive level of the subject (u1) based on the staying time.

According to this configuration, the cognitive level is evaluated based on the staying time. When there is a possibility that the subject has the dementia or the MCI, the staying time in the toilet space 5 tends to be changed. Thus, the cognitive level can be relatively easily evaluated by using the staying time in the toilet space (5).

In a cognitive level evaluation system (1) according to a sixth aspect, which may be implemented in conjunction with the first aspect, the detector (141) is configured to detect a time required for the behavior of the subject (u1) in the toilet space (5). The cognitive level evaluator (143) is configured to evaluate the cognitive level of the subject (u1) based on the time required for the behavior.

According to this aspect, the cognitive level is evaluated based on the time required for the behavior. When there is a possibility that the subject has the dementia or the MCI, the time required for the behavior in the toilet space 5 may be changed. Thus, the cognitive level can be relatively easily evaluated by using the time required for the behavior in the toilet space (5).

In a cognitive level evaluation system (1) according to a seventh aspect, which may be implemented in conjunction with the first aspect, the detector (141) is configured to detect a kind of the behavior of the subject (u1) in the toilet space (5). The cognitive level evaluator (143) is configured to evaluate the cognitive level of the subject (u1) based on the kind of the behavior.

According to this configuration, the cognitive level is evaluated based on the kind of the behavior. When there is a possibility that the subject has the dementia or the MCI, the kind of the behavior in the toilet space 5 may be changed. Thus, the cognitive level can be relatively easily evaluated by using the kind of the behavior in the toilet space (5).

In a cognitive level evaluation system (1) according to an eighth aspect, which may be implemented in conjunction with the first aspect, the detector (141) is configured to detect a plurality of the behaviors of the subject (u1) in the toilet space (5). The cognitive level evaluator (143) is configured to evaluate the cognitive level of the subject (u1) based on whether or not the plurality of the behaviors include overlapping behaviors.

According to this configuration, the cognitive level is evaluated based on whether or not the plurality of the behaviors include the overlapping behaviors. When there is a possibility that the subject has the dementia or the MCI, the subject may perform the overlapping behaviors. Thus, the cognitive level can be relatively easily evaluated by using the result about whether or not the plurality of the behaviors include the overlapping behaviors.

In a cognitive level evaluation system (1) according to a ninth aspect, which may be implemented in conjunction with the first aspect, the detector (141) is configured to detect an operation input to an operation device (22), as the behavior of the subject (u1) in the toilet space (5). The cognitive level evaluator (143) is configured to evaluate the cognitive level of the subject (u1) based on the operation.

According to this configuration, the cognitive level is evaluated based on the operation input to the operation device (22). When there is a possibility that the subject has the dementia or the MCI, a change may occur in the subject's operation to the operation device (22) in the toilet space (5). Thus, the cognitive level can be evaluated in accordance with the operation to the operation device (22) in the toilet space (5).

In a cognitive level evaluation system (1) according to a tenth aspect, which may be implemented in conjunction with the ninth aspect, the cognitive level evaluator (143) is configured to evaluate the cognitive level of the subject (u1) based on a rate of long pressing operation to a push button (operation part B10) provided for the operation device (22).

According to this configuration, the cognitive level is evaluated based on the rate of long pressing operation. When there is a possibility that the subject has the dementia or the MCI, a change may occur in the operation to the push button. Thus, the cognitive level can be evaluated in accordance with the rate of long pressing operation with respect to the push button of the operation device (22).

In a cognitive level evaluation system (1) according to an eleventh aspect, which may be implemented in conjunction with the ninth aspect, the cognitive level evaluator (143) is configured to evaluate the cognitive level of the subject (u1) based on consecutive operation to a push button (operation part B10) provided for the operation device (22).

According to this configuration, the cognitive level is evaluated based on the consecutive operation to the push button. When there is a possibility that the subject has the dementia or the MCI, a change may occur in the operation to the push button. Thus, the cognitive level can be evaluated in accordance with the consecutive operation to the push button of the operation device (22).

In a cognitive level evaluation system (1) according to a twelfth aspect, which may be implemented in conjunction with the first aspect, the cognitive level evaluator (143) is configured to evaluate the cognitive level of the subject (u1) based on at least one of: a time of day when the behavior of the subject (u1) is detected in the toilet space (5) by the detector (141); or a detecting frequency at which the behavior of the subject (u1) is detected in the toilet space (5) by the detector (141).

According to this configuration, the cognitive level is evaluated based on at least one of: the time of day when the behavior of the subject (u1) is detected in the toilet space (5) by the detector (141); or the detecting frequency at which the behavior of the subject (u1) is detected in the toilet space (5) by the detector (141). When there is a possibility that the subject has the dementia or the MCI, a change may occur in a time of day when the toilet space (5) is used or a frequency at which the toilet space (5) is used. Thus, the cognitive level can be relatively easily evaluated by using at least one of: the time of day when the behavior of the subject (u1) is detected in the toilet space (5) by the detector (141); or the detecting frequency at which the behavior of the subject (u1) is detected in the toilet space (5) by the detector (141).

In a cognitive level evaluation system (1) according to a thirteenth aspect, which may be implemented in conjunction with the first aspect, the detector (141) is configured to detect presence or absence of excrement of the subject (u1), as the behavior of the subject (u1) in the toilet space (5). The cognitive level evaluator (143) is configured to evaluate the cognitive level of the subject (u1) based on the presence or absence of excrement of the subject (u1).

According to this configuration, the cognitive level is evaluated based on the presence or absence of excrement of the subject (u1). When there is a possibility that the subject has the dementia or the MCI, the subject may leave the toilet space (5) without performing an excretion action. Thus, the cognitive level can be relatively easily evaluated by using the presence or absence of excrement.

A cognitive level evaluation system (1) according to a fourteenth aspect, which may be implemented in conjunction with any one of the first to thirteenth aspects, further includes a reporting unit (144). The reporting unit (144) is configured to report, when finding that lacking behavior in the toilet space (5) is present, lack behavior information about the lacking behavior.

This configuration can contribute making a person aware of the lacking behavior in the toilet space (5).

A cognitive level evaluation method according to a fifteenth aspect includes a detecting step and a cognitive level evaluating step. The detecting step includes detecting a behavior of a subject in a toilet space (5). The cognitive level evaluating step includes evaluating a cognitive level of the subject based on a detection result in the detecting step.

This cognitive level evaluation method can realize evaluating the cognitive level of the subject based on the subject's behavior in the toilet space (5), as one aspect of life.

A program according to a sixteenth aspect is designed to cause a computer to execute the cognitive level evaluation method of the fifteenth aspect.

This program can realize evaluating the cognitive level of the subject based on the subject's behavior in the toilet space (5), as one aspect of life.

### REFERENCE SIGNS LIST

1 Cognitive Level Evaluation System
5 Toilet Space
10 Evaluation Device
22 Operation Device
141 Detector
143 Cognitive level evaluator
144 Reporting Unit
B10 Operation Part

1. A cognitive level evaluation system, comprising:
a detector configured to detect a behavior of a subject in a toilet space; and
a cognitive level evaluator configured to evaluate a cognitive level of the subject based on a detection result of the detector.

2. The cognitive level evaluation system of claim 1, wherein
the detector is configured to detect a plurality of the behaviors of the subject in the toilet space, and
the cognitive level evaluator is configured to evaluate the cognitive level of the subject based on an order of the plurality of the behaviors.

3. The cognitive level evaluation system of claim 2, wherein
the cognitive level evaluator is configured to evaluate the cognitive level of the subject by comparing the order of the plurality of the behaviors detected by the detector with a behavioral order previously memorized, of the subject in the toilet space.

4. The cognitive level evaluation system of claim 1, wherein
the cognitive level evaluator is configured to evaluate the cognitive level of the subject based on whether or not the behavior of the subject is detected in the toilet space by the detector.

5. The cognitive level evaluation system of claim 1, wherein
the detector is configured to detect a staying time of the subject staying in the to space, as the behavior of the subject in the toilet space, and
the cognitive level evaluator is configured to evaluate the cognitive level of the subject based on the staying time.

6. The cognitive level evaluation system of claim 1, wherein
the detector is configured to detect a time required for the behavior of the subject in the toilet space, and
the cognitive level evaluator is configured to evaluate the cognitive level of the subject based on the time required for the behavior.

7. The cognitive level evaluation system of claim 1, wherein
the detector is configured to detect a kind of the behavior of the subject in the toilet space, and
the cognitive level evaluator is configured to evaluate the cognitive level of the subject based on the kind of the behavior.

8. The cognitive level evaluation system of claim 1, wherein
the detector is configured to detect a plurality of the behaviors of the subject in the toilet space, and
the cognitive level evaluator is configured to evaluate the cognitive level of the subject based on whether or not the plurality of the behaviors include overlapping behaviors.

9. The cognitive level evaluation system of claim 1, wherein
the detector is configured to detect an operation input to an operation device, as the behavior of the subject in the toilet space, and
the cognitive level evaluator is configured to evaluate the cognitive level of the subject based on the operation.

10. The cognitive level evaluation system of claim 9, wherein
the cognitive level evaluator is configured to evaluate the cognitive level of the subject based on a rate of long pressing operation to a push button provided for the operation device.

11. The cognitive level evaluation system of claim 9, wherein
the cognitive level evaluator is configured to evaluate the cognitive level of the subject based on consecutive operation to a push button provided for the operation device.

12. The cognitive level evaluation system of claim 1, wherein
the cognitive level evaluator is configured to evaluate the cognitive level of the subject based on at least one of:
a time of day when the behavior of the subject is detected in the toilet space by the detector; or
a detecting frequency at which the behavior of the subject is detected in the toilet space by the detector.

13. The cognitive level evaluation system of claim 1, wherein
the detector is configured to detect presence or absence of excrement of the subject, as the behavior of the subject in the toilet space, and
the cognitive level evaluator is configured to evaluate the cognitive level of the subject based on the presence or absence of excrement of the subject.

14. The cognitive level evaluation system of claim 1, further comprising a reporting unit, wherein
the reporting unit is configured to report, when finding that lacking behavior in the toilet space is present, lack behavior information about the lacking behavior.

15. A cognitive level evaluation method, comprising:
a detecting step including detecting a behavior of a subject in a toilet space; and
a cognitive level evaluating step including evaluating a cognitive level of the subject based on a detection result in the detecting step.

16. A non-transitory computer-readable tangible recording medium storing a program designed to cause a computer to execute the cognitive level evaluation method of claim 15.
